(19) United States
(12) Patent Application Publication
(10) Pub. No.: US 2024/0366703 A1
(43) Pub. Date: Nov. 7, 2024

(54) DIETARY SUPPLEMENT COMPOSITIONS AND METHODS FOR THEIR USE TO IMPROVE WORKOUT PERFORMANCE

(71) Applicant: Melaleuca, Inc., Idaho Falls, ID (US)

(72) Inventors: Jordan Joy, Idaho Falls, ID (US); Nasser A. Fredj, Idaho Falls, ID (US)

(21) Appl. No.: 18/654,418

(22) Filed: May 3, 2024

Related U.S. Application Data
(60) Provisional application No. 63/463,721, filed on May 3, 2023.

(51) Int. Cl. A61K 36/31; A61K 9/00; A61K 31/197; A61K 31/4415; A61K 31/455; A61K 31/51; A61K 31/522; A61K 31/525; A61K 31/714; A61K 36/22; A61K 36/87

(52) U.S. Cl. CPC A61K 36/31; A61K 9/0095; A61K 31/197; A61K 31/4415; A61K 31/455; A61K 31/51; A61K 31/522; A61K 31/525; A61K 31/714; A61K 36/22; A61K 36/87

(57) ABSTRACT

Dietary supplement compositions are provided herein. Also provided herein arc methods for using the dietary supplement compositions as pre-workout supplements or as supplements during a workout.

### CROSS-REFERENCE TO RELATED APPLICATIONS

This application claims priority from U.S. Provisional Application Ser. No. 63/463,721, filed May 3, 2023. The disclosure of the prior application is considered part of (and is incorporated by reference in) the disclosure of this application.

### TECHNICAL FIELD

This document relates to dietary supplements. For example, this document relates to dietary supplement compositions that can be consumed before or during a physical workout, and to methods of using the dietary supplements described herein to improve performance during a physical workout.

### BACKGROUND

Consumers in the fitness and wellness space often desire supplements prior to training that would enhance energy and workout performance.

### SUMMARY

This document provides dietary supplement compositions that can contain arugula extract, mango leaf extract, and extracts from grape seed, grape skin, or both grape seed and grape skin. In some cases, the dietary supplement compositions also can contain caffeine and/or one or more B vitamins. This document also provides methods for using the compositions described herein. In some cases, the compositions can be used as pre-workout supplements, or as supplements consumed during a workout. The compositions described herein can, for example, enhance energy and workout (or physical training) performance, especially when consumed before or during a workout. Thus, the compositions can benefit consumers in the fitness and wellness space and can provide enhanced performance and long term progress. The compositions described herein can result in acute (single dose) and/or longitudinal (several weeks to months) improvement in performance and/or body composition goals.

In a first aspect, this document features a composition containing, consisting of, or consisting essentially of arugula extract; grape seed extract, grape skin extract, or a combination thereof; and mango leaf extract. The composition can contain about 20% to about 50% by weight of the arugula extract. The composition can contain the arugula extract in an amount sufficient to produce about 90 mg to about 1240 mg nitrate (e.g., about 300 mg to about 400 mg nitrate). The composition can contain the arugula extract in an amount of about 2 g to about 15 g, such as, about 3.6 g to about 4.8 g. The composition can contain about 2% to about 5% by weight of the grape seed extract, grape skin extract, or combination thereof. The composition can contain the grape seed extract, grape skin extract, or combination thereof in an amount of about 100 mg to about 500 mg, such as, about 200 mg to about 400 mg. The composition can contain about 2% to about 5% by weight of the mango leaf extract. The composition can contain the mango leaf extract in an amount of about 140 mg to about 19,350 mg, such as, about 200 mg to about 400 mg.

In some cases, the composition can further contain caffeine. The composition can contain about 1% to about 5% by weight of the caffeine. The composition can contain the caffeine in an amount of about 18 mg to about 540 mg, such as, about 180 mg to about 360 mg.

In some cases, the composition can further contain at least one B vitamin. The at least one B vitamin can be selected from the group consisting of: thiamin, riboflavin, niacin, vitamin B6, vitamin B12, and pantothenic acid. The composition can contain one or more of: about 0.01% to about 0.05% by weight of thiamin, about 0.01% to about 0.05% by weight of riboflavin, about 0.05% to about 0.25% by weight of niacin, about 0.005% to about 0.05% by weight of B6, about 0.001% to about 0.01% by weight of B12, and about 0.00056% to about 0.56% by weight of pantothenic acid. The composition can contain one or more of: thiamin in an amount of about 0.12 mg to about 12 mg, riboflavin in an amount of about 0.13 mg to about 13 mg, niacin in an amount of about 1.6 mg NE to about 80 mg NE, B6 in an amount of about 0.17 mg to about 170 mg, B12 in an amount of about 0.00024 mg to about 4.8 mg, and pantothenic acid in an amount of about 0.05 mg to about 50 mg. In some cases, the composition can contain one or more of: thiamin in an amount of about 0.6 mg to about 2.4 mg, riboflavin in an amount of about 0.65 mg to about 2.6 mg, niacin in an amount of about 8 mg NE to about 32 mg NE, B6 in an amount of about 0.3 mg to about 3.2 mg, B12 in an amount of about 0.0012 mg to about 0.0048 mg, and pantothenic acid in an amount of about 2.5 mg to about 10 mg.

In some cases, the composition can contain (a) arugula extract; (b) grape seed extract, grape skin extract, or combination thereof; (c) mango leaf extract; (d) caffeine; and (e) one or more B vitamins.

In some cases, the composition can further contain at least one acidifier. The at least one acidifier can be selected from the group consisting of: citric acid, malic acid potassium citrate, and citrulline malate.

In some cases, the composition can further contain at least one sweetener. The at least one sweetener can be selected from the group consisting of: stevia, sucralose, acesulfame potassium, aspartame, monk fruit sugar, dextrose, sucrose, fructose, glucose, maltose, cane sugar, sugar alcohol (e.g., erythritol, maltitol, or xylitol), and rare sugar (e.g., allulose or tagatose).

In some cases, the composition can further contain a thickener. The thickener can be guar gum, xanthan gum, locust bean gum, or cellulose gum. In some cases, the composition can further contain one or more natural flavors and/or one or more colorants.

In some cases, the composition can be formulated as a solid (e.g., a powder). In some cases, the composition can be formulated as a liquid.

This document also features a method for increasing energy and workout performance in a subject. The method can include administering to the subject a composition as described herein. The method can include activating muscles in the subject. The composition can be effective to reduce oxygen cost and increase oxygen supply in muscles and brain of the subject. The composition can be administered to the subject prior to physical training (e.g., about 30 minutes to about 120 minutes prior to physical training). In some cases, the composition can be administered to the subject during physical training. The subject can be a human.

In another aspect, this document features a powdered dietary supplement composition contained in a bulk package, the bulk package containing a measuring scoop and the powdered dietary supplement composition, the measuring scoop being sized to scoop an amount of the powdered dietary supplement composition suitable for blending with a liquid to form a beverage, wherein the amount of the powdered dietary supplement composition is 15 g to 40 g and contains, consists of, or consists essentially of (a) arugula extract in an amount of about 20 wt % to about 50 wt %; (b) grape seed extract, grape skin extract, or a combination thereof, in an amount of about 2 wt % to about 5 wt %; and (c) mango leaf extract in an amount of about 2 wt % to about 5 wt %. The powdered dietary supplement composition can further contain caffeine in an amount of about 1 wt % to about 5 wt %. The powdered dietary supplement can further contain at least one B vitamin.

As used herein, the term “about” when used to refer to weight % in a composition means ±10% of the reported weight %. As used herein, the term “about” when used to refer to measured characteristics of the composition means ±20% of the reported value.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and from the claims.

### DETAILED DESCRIPTION

Described herein are dietary supplement compositions that contain arugula, mango leaf extract, and extracts from grape seed, grape skin, or both grape seed and grape skin. In some cases, one or more B vitamins can be included in the compositions. In some cases, the compositions can contain caffeine. In some cases, the compositions can contain one or more additional ingredients, as described herein. Also described herein is the use of the compositions. In some cases, the compositions can be used as pre-workout supplements. For example, the compositions described herein can enhance energy and workout (or physical training) performance, especially when consumed before a workout but also, at least in some cases, when consumed during a workout. Thus, the compositions can benefit consumers in the fitness and wellness space and can provide enhanced performance and long term progress, in particular, with repeated and consistent use. The compositions described herein can result in acute (single dose) and/or longitudinal (several weeks to months) improvement in performance and/or body composition goals.

### Dietary Compositions

The dietary supplement compositions described herein can include arugula extract; grape seed extract, grape skin extract, or a combination of both (e.g., a blend of grape seed and skin extract); and mango leaf extract. In some cases, the compositions can include one or more additional ingredients. For example, the compositions can contain caffeine, one or more B vitamins (e.g., one or more of thiamin, riboflavin, niacin, B6, B12, and pantothenic acid), one or more acidifiers (e.g., one or more of citric acid, malic acid potassium citrate, and citrulline malate), one or more sweeteners (e.g., one or more of stevia, sucralose, acesulfame potassium, aspartame, monk fruit sugar, dextrose, sucrose, fructose, glucose, maltose, cane sugar, sugar alcohol (e.g., erythritol, maltitol, and/or xylitol), and rare sugar (e.g., allulose and/or tagatose)), one or more thickeners (e.g., guar gum, xanthan gum, locust bean gum, and/or cellulose gum), one or more colorants, and/or one or more natural flavors.

### Arugula Extract (and Other Sources of Nitrate)

The compositions described herein can contain arugula extract. Arugula extract is a source of dietary nitrates that can be reduced to nitric oxide (NO). Without being bound by a particular mechanism, nitric oxide and nitrates can improve muscular contraction efficiency by reducing ATP and oxygen cost. Additionally, or in the alternative, nitric oxide and nitrates can improve short and long duration performance, such as strength endurance, power output, and race performance.

In some cases, the compositions provided herein can contain one or more nitrate sources in addition to or in place of arugula extract. For example, dietary supplement compositions can contain extracts from other nitrate-containing plants. Such nitrate sources include, without limitation celery, beets, spinach, red spinach, citrulline, arginine, corn, sorghum, oat hay, johnsongrass, carelessweed, kochia, pigweed, Russian thistle, nightshade, lettuce, fennel, radishes, parsley, potatoes, sweet potatoes, carrots, radishes, onions, yams, and Chinese cabbage. In some cases, dietary supplement compositions can contain one or more amino acids (e.g., amino acids used to form nitric oxide via nitric oxide synthase). In some cases, the compositions described here can include arugula extract and one or more other nitrate sources such as those listed above. In some cases, the compositions can contain one or more of the other nitrate sources instead of arugula extract (e.g., one or more of the other nitrate sources can be present in the compositions in the absence of arugula extract).

In some cases, compositions described herein can include about 20% to about 50% (e.g., about 20% to about 25%, about 25% to about 30%, about 30% to about 35%, about 35% to about 40%, about 40% to about 45%, or about 45% to about 50%) by weight of arugula extract (and/or one or more other nitrate sources such as those described herein). For example, a composition described herein can include about 40% to about 50% (e.g., about 43%, about 45%, or about 47%) by weight of arugula extract (and/or one or more other nitrate sources described above).

In some cases, compositions described herein can include arugula (Eruca sativa) extract (and/or one or more of the other nitrate sources described above) sufficient to produce about 90 mg to about 1240 mg (e.g., about 90 mg to about 100 mg, about 100 mg to about 200 mg, about 200 mg to about 300 mg, about 300 mg to about 400 mg, about 400 mg to about 500 mg, about 500 mg to about 600 mg, about 600 mg to about 700 mg, about 700 mg to about 800 mg, about 800 mg to about 900 mg, about 900 mg to about 1000 mg, about 1000 mg to about 1100 mg, about 1100 mg to about 1200 mg, about 1200 mg to about 1240 mg) nitrate per serving of the composition. For example, a single serving of a composition described herein can include arugula extract (and/or one or more of the other nitrate sources described above) in an amount that is sufficient to produce about 90 mg to about 1240 mg (e.g., about 300 mg to about 400 mg, such as about 300 to about 330 mg, about 310 to about 340 mg, or about 316 mg) nitrate, when the single serving of the composition is consumed by a subject (e.g., prior to or during physical training or workout). In some cases, compositions described herein can include arugula extract (and/or one or more of the other nitrate sources described above) in an amount of about 1 g to about 15 g per serving (e.g., about 1 g to about 1.5 g, about 1.5 g to about 2 g, about 1 g to about 2 g, about 1 g to about 3 g, about 2 g to about 3 g, about 3 g to about 5 g, about 5 g to about 7 g, about 7 g to about 9 g, about 9 g to about 11 g, about 11 g to about 13 g, or about 13 g to about 15 g per serving). For example, a single serving of a composition described herein can include arugula extract (and/or one or more of the other nitrate sources described above) in an amount of about 3.6 g to about 4.8 g, such as about 3.7 g, about 3.8 g, or about 3.9 g.

Grape Seed and/or Grape Skin Extract

The compositions described herein can contain grape seed extract, grape skin extract, or a combination of both grape seed extract and grape skin extract. For example, the compositions can contain a blend of grape seed and grape skin extracts. In some cases, the compositions can contain a pre-mixed combination of grape seed extract and/or grape skin extract with additional ingredients (e.g., as in PROVEXCV®; Melaleuca, Inc., Idaho Falls, ID). Without being bound by a particular mechanism, grape skin and/or grape skin extract can reduce myeloperoxidase (MPO) activity to facilitate natural nitric oxide production through endothelial nitric oxide synthase (eNOS). Nitric oxide and nitrates can improve muscular contraction efficiency by reducing ATP and oxygen cost, and can improve short and long duration performance (e.g., strength endurance, power output, race performance).

In some cases, compositions described herein can include about 2.0% to about 5.0% (e.g., about 2.0% to about 2.5%, about 2.5% to about 3.0%, about 3.0% to about 3.5%, about 3.5% to about 4.0%, about 4.0% to about 4.5%, or about 4.5% to about 5.0%) by weight of grape skin and/or grape skin extract. For example, a composition described herein can include about 3.0% to about 4.0% (e.g., about 3.4%, about 3.6%, or about 3.8%) by weight of grape skin extract, grape seed extract, or a combination thereof.

In some cases, compositions described herein can include grape skin and/or grape skin extract in an amount of about 100 mg to about 500 mg per serving (e.g., about 100 mg to about 200 mg, about 200 mg to about 300 mg, about 300 mg to about 400 mg, or about 400 mg to about 500 mg per serving). For example, a single serving of a composition described herein can include grape skin extract, grape seed extract, or a combination thereof in an amount of about 200 mg to about 400 mg, such as about 300 mg, about 330 mg, or about 350 mg.

### Mango Leaf Extract

Compositions described herein can contain mango (Mangifera indica) leaf extract. Mango leaf extract can serve as a source of an antioxidant, mangiferin, which can help with muscle and brain oxygenation. Without being bound by a particular mechanism, the mango leaf extract can work synergistically with nitrates (e.g., nitrates produced by arugula extract and/or grape seed and grape skin extract) to improve aerobic exercise performance, e.g., by complementing the reduction in oxygen cost. Additionally, or in the alternative, mango leaf extract can enhance brain oxygenation, and the increased oxygen supply to the brain can facilitate improved cognitive function.

The mango leaf extract present in the compositions provided herein can contain mangiferin, which is a flavonoid (in particular, a xanthone). In some cases, the compositions described here can include one or more other flavonoids, such as one or more xanthones, flavanols, flavones, anthocyanidins, flavanones, and/or isoflavones. Flavonoids that can be present in the compositions include, without limitation, one or more of mangiferin, quercetin, luteolin, kaempferol, myricetin, fisetin, isorhamnetin, catechin (e.g., epicatechin, epigallocatechin, gallocatechin), proanthocyanidins, theaflavins, thearubigins, apigenin, baicalein, chrysin, mangostin (e.g., alpha-mangostin, beta-mangostin, and/or gamma-mangostin), gambogic acid, garcinone A through E, gartanin, and 8-desoxygartanin. In some cases, the compositions described here can include mango leaf extract and one or more additional flavonoids (e.g., one or more of the flavonoids described herein, such as luteolin). In some cases, the compositions can contain one or more flavonoids instead of mango leaf extract (e.g., one or more of the flavonoids listed above can be present in the compositions in the absence of mango leaf extract).

In some cases, compositions described herein can include about 2.0% to about 5.0% (e.g., about 2.0% to about 2.5%, about 2.5% to about 3.0%, about 3.0% to about 3.5%, about 3.5% to about 4.0%, about 4.0% to about 4.5%, or about 4.5% to about 5.0%) by weight of mango leaf extract (and/or one or more flavonoids described above). For example, a composition described herein can include about 3.0% to about 4.0% (e.g., about 3.2%, about 3.5%, or about 3.8%) by weight of mango leaf extract (and/or one or more flavonoids described above).

In some cases, compositions described herein can include mango leaf extract (and/or one or more flavonoids described above) in an amount of about 140 mg to about 19,500 mg (e.g., about 140 mg to about 200 mg, about 200 mg to about 400 mg, about 400 mg to about 600 mg, about 600 mg to about 800 mg, about 800 mg to about 1000 mg, about 1000 mg to about 2000 mg, about 2000 mg to about 3000 mg, about 3000 mg to about 4000 mg, about 4000 mg to about 5000 mg, about 5000 mg to about 6000 mg, about 6000 mg to about 7000 mg, about 7000 mg to about 8000 mg, about 8000 mg to about 9000 mg, about 9000 mg to about 10000 mg, about 10000 mg to about 12000 mg, about 12000 mg to about 14000 mg, about 14000 mg to about 16000 mg, about 16000 mg to about 18000 mg, about 18000 mg to about 18500 mg, or about 18500 mg to about 19500 mg) per serving. For example, a single serving of a composition described herein can include mango leaf extract (and/or one or more flavonoids described above) in an amount of about 200 mg to about 400 mg, such as about 250 mg, about 300 mg, or about 350 mg.

In some cases, dietary supplement compositions can contain a combination of mango leaf extract (or mangiferin) and another flavonoid (e.g., luteolin). The other flavonoid can be present in any appropriate amount. For example, dietary supplement compositions can contain mango leaf extract in an amount as described above, and another flavonoid (e.g., luteolin and/or quercetin in an amount of about 5 mg to about 500 mg per serving (e.g., about 5 mg to about 50 mg, about 50 mg to about 100 mg, about 100 mg to about 500 mg, or about 50 mg per serving). The mango leaf extract (or mangiferin) and the other flavonoid(s) can be present in the dietary supplement compositions in any appropriate ratio (e.g., about 5:1 mango leaf extract to luteolin or other flavonoid, about 6:1 mango leaf extract to luteolin or other flavonoid, about 1:1 mango leaf extract to quercetin or other flavonoid, about 18:5 mangiferin to luteolin, or about 21:35 mangiferin to quercetin). In some cases, dietary supplement compositions can contain mango leaf extract and quercetin, where the quercetin is present in an amount of about 60 mg to about 6000 mg per serving (e.g., about 60 mg to about 300 mg, about 300 mg to about 500 mg, about 500 mg to about 700 mg, about 700 mg to about 1000 mg, about 1000 mg to about 6000 mg, or about 600 mg per serving). In some cases, tiger nut extract can be included in the dietary supplement compositions provided herein (e.g., in an amount of about 35 mg to about 3500 mg per serving (e.g., about 35 mg to about 250 mg, about 250 mg to about 450 mg, about 450 mg to about 1000 mg, about 1000 mg to about 3500 mg, or about 350 mg per serving). In some cases, dietary supplement compositions can contain mango extract, quercetin, and tiger nut extract in a ratio of about 6:12:7 mango: quercetin: tiger nut. In some cases, dietary supplement compositions can contain mangiferin, quercetin, and tiger nut extract in a ratio of about 18:60:35 mangiferin: quercetin: tiger nut.

### Caffeine

In some cases, the compositions described herein can contain caffeine (e.g., caffeine sourced from coffee beans (e.g., green coffee beans), tea (e.g., green tea), dicaffeine maleate, guayusa, or any other suitable source). Without being bound by a particular mechanism, caffeine can improve attention, reaction time, power, strength, endurance, and/or sport skill performance. In some cases, caffeine can function to block adenosine receptors to prevent fatigue, promote release of adrenaline to increase lipolysis and nutrient availability for aerobic respiration, and promote release of calcium within muscles to improve muscle contractile strength.

In some cases, compositions described herein can include about 1.0% to about 5.0% (e.g., about 1.0% to about 1.5%, about 1.5% to about 2.0%, about 2.0% to about 2.5%, about 2.5% to about 3.0%, about 3.0% to about 3.5%, about 3.5% to about 4.0%, about 4.0% to about 4.5%, or about 4.5% to about 5.0%) by weight of caffeine. For example, a composition described herein can include about 2.0% to about 3.0% (e.g., about 2.2%, about 2.3%, or about 2.4%) by weight of caffeine.

In some cases, compositions described herein can contain caffeine in an amount of about 18 mg to about 540 mg (e.g., about 18 mg to about 20 mg, about 20 mg to about 40 mg, about 40 mg to about 60 mg, about 60 mg to about 80 mg, about 80 mg to about 100 mg, about 100 mg to about 120 mg, about 120 mg to about 140 mg, about 140 mg to about 160 mg, about 160 mg to about 180 mg, about 180 mg to about 200 mg, about 200 mg to about 220 mg, about 220 mg to about 240 mg, about 240 mg to about 260 mg, about 260 mg to about 280 mg, about 280 mg to about 300 mg, about 300 mg to about 320 mg, about 320 mg to about 340 mg, about 340 mg to about 360 mg, about 360 mg to about 380 mg, about 380 mg to about 400 mg, about 400 mg to about 420 mg, about 420 mg to about 440 mg, about 440 mg to about 460 mg, about 460 mg to about 480 mg, about 480 mg to about 500 mg, about 500 mg to about 520 mg, or about 520 mg to about 540 mg) per serving. For example, a single serving of a composition described herein can include caffeine in an amount of about 180 mg to about 360 mg, such as about 180 mg.

### B Vitamins

The compositions described herein can contain one or more B vitamins, such as one or more of thiamin, riboflavin, niacin, B6, B12, and pantothenic acid. Without being bound by a particular mechanism, B vitamins can act as cofactors in nutrient metabolism and ATP production, and thus can support energy metabolism.

### Thiamin (Vitamin B1)

In some cases, compositions described herein can include thiamin (also known as vitamin B1). The thiamin can be present as, for example, thiamin minonitrate, thiamin hydrocholoride, or benfotiamine. In some cases, compositions described herein can include about 0.01% to about 0.05% (e.g., about 0.01% to about 0.02%, about 0.02% to about 0.03%, about 0.03% to about 0.04%, or about 0.04% to about 0.05%) by weight of thiamin. For example, a composition described herein can include about 0.01% to about 0.03% (e.g., about 0.02%) by weight of thiamin.

In some cases, compositions described herein can include thiamin in an amount of about 0.12 mg to about 12 mg per serving (e.g., about 0.12 mg to about 0.15 mg, about 0.15 mg to about 0.20 mg, about 0.20 mg to about 0.30 mg, about 0.30 mg to about 0.40 mg, about 0.4 mg to about 0.5 mg, about 0.5 mg to about 0.6 mg, about 0.6 mg to about 0.7 mg, about 0.7 mg to about 0.8 mg, about 0.8 mg to about 0.9 mg, about 0.9 mg to about 1 mg, about 1 mg to about 2 mg, about 2 mg to about 4 mg, about 4 mg to about 6 mg, about 6 mg to about 8 mg, about 8 mg to about 10 mg, or about 10 mg to about 12 mg per serving). For example, a single serving of a composition described herein can include thiamin in an amount of about 0.6 mg to about 2.4 mg, such as about 0.8 mg, about 1.0 mg, or about 1.2 mg.

### Riboflavin (Vitamin B2)

In some cases, the compositions described herein can include riboflavin (also known as vitamin B2). The riboflavin can be present as flavin adenine dinucleotide (FAD) and/or flavin mononucleotide (FMN).

In some cases, compositions described herein can include about 0.01% to about 0.05% (e.g., about 0.01% to about 0.02%, about 0.02% to about 0.03%, about 0.03% to about 0.04%, or about 0.04% to about 0.05%) by weight of riboflavin. For example, a composition described herein can include about 0.01% to about 0.03% (e.g., about 0.02%) by weight of riboflavin.

In some cases, compositions described herein can include riboflavin in an amount of about 0.13 mg to about 13 mg (e.g., about 0.13 mg to about 0.15 mg, about 0.15 mg to about 0.3 mg, about 0.3 mg to about 0.5 mg, about 0.5 mg to about 1 mg, about 1 mg to about 3 mg, about 3 mg to about 5 mg, about 5 mg to about 7 mg, about 7 mg to about 9 mg, about 9 mg to about 11 mg, or about 11 mg to about 13 mg) per serving. For example, a single serving of a composition described herein can include riboflavin in an amount of about 0.65 mg to about 2.60 mg, such as about 0.8 mg, about 1 mg, or about 1.2 mg.

### Niacin (Vitamin B3)

In some cases, the compositions described herein can include niacin (also known as vitamin B3). The niacin can be present as, for example, niacinamide, nicotinamide, nicotinamide riboside, or nicotinic acid.

In some cases, compositions described herein can include about 0.05% to about 0.25% (e.g., about 0.05% to about 0.10%, about 0.10% to about 0.15%, about 0.15% to about 0.20%, or about 0.20% to about 0.25%) by weight of niacin. For example, a composition described herein can include about 0.10% to about 0.20%, (e.g., about 0.15%) by weight of niacin.

In some cases, compositions described herein can include niacin in an amount of about 1.6 mg niacin equivalents (NE) to about 80 mg NE (e.g., about 1.6 mg NE to about 2 mg NE, about 2 mg NE to about 4 mg NE, about 4 mg NE to about 6 mg NE, about 6 mg NE to about 8 mg NE, about 8 mg NE to about 10 mg NE, about 10 mg NE to about 20 mg NE, about 20 mg NE to about 30 mg NE, about 30 mg NE to about 40 mg NE, about 40 mg NE to about 50 mg NE, about 50 mg NE to about 60 mg NE, about 60 mg NE to about 70 mg NE, or about 70 mg NE to about 80 mg NE) per serving. For example, a single serving of a composition described herein can include niacin in an amount of about 8 mg NE to about 32 mg NE, such as about 8 mg NE, about 10 mg NE, to about 12 mg NE.

### Pyridoxine (Vitamin B6)

In some cases, the compositions described herein can include pyridoxine (also known as vitamin B6). The pyridoxine can be present as pyridoxal 5′ phosphate (PLP), pyridoxal, pyridoxamine, and/or B6 hydrochloride.

In some cases, compositions described herein can include about 0.005% to about 0.05% (e.g., about 0.005% to about 0.01%, about 0.01% to about 0.015%, about 0.015% to about 0.02%, about 0.02% to about 0.025%, about 0.025% to about 0.03%, about 0.03% to about 0.035%, about 0.035% to about 0.04%, about 0.04% to about 0.045%, or about 0.045% to about 0.05%) by weight of pyridoxine (vitamin B6). For example, a composition described herein can include about 0.005% to about 0.015%, (e.g., about 0.01%) by weight of pyridoxine (vitamin B6).

In some cases, compositions described herein can include pyridoxine (vitamin B6) in an amount of about 0.17 mg to about 170 mg (e.g., about 0.17 mg to about 0.5 mg, about 0.5 mg to about 0.75 mg, about 0.75 mg to about 1 mg, about 1 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 60 mg, about 60 mg to about 70 mg, about 70 mg to about 80 mg, about 80 mg to about 90 mg, about 90 mg to about 100 mg, about 100 mg to about 110 mg, about 110 mg to about 120 mg, about 120 mg to about 130 mg, about 130 mg to about 140 mg, about 140 mg to about 150 mg, about 150 mg to about 160 mg, or about 160 mg to about 170 mg) per serving. For example, a single serving of a composition described herein can include pyridoxine (vitamin B6) in an amount of about 0.3 mg to about 3.2 mg (e.g., about 0.4 mg, about 0.5 mg, about 1 mg, about 2 mg, about 3 mg).

### Cobalamin (Vitamin B12)

In some cases, compositions described herein include cobalamin (also known as vitamin B12). Cobalamin (vitamin B12) can be present as cyanocobalamin or methylcobalamin.

In some cases, compositions described herein include about 0.001% to about 0.01% (e.g., about 0.001% to about 0.002%, about 0.002% to about 0.003%, about 0.003% to about 0.004%, about 0.004% to about 0.005%, or about 0.005% to about 0.008%, or about 0.008% to about 0.01%) by weight of cobalamin (vitamin B12). For example, a composition described herein can include about 0.001% by weight of cobalamin (vitamin B12).

In some cases, compositions described herein include cobalamin (vitamin B12) in an amount of about 0.00024 mg to about 4.8 mg (e.g., about 0.00024 mg to about 0.00050 mg, about 0.00050 mg to about 0.0010 mg, about 0.0010 mg to about 0.0050 mg, about 0.0050 mg to about 0.010 mg, about 0.010 mg to about 0.05 mg, about 0.05 mg to about 0.10 mg, about 0.10 mg to about 0.50 mg, about 0.50 mg to about 1.0 mg, about 1.0 mg to about 1.2 mg, about 1.2 mg to about 2.4 mg, about 2.4 mg to about 3.6 mg, or about 3.6 mg to about 4.8 mg) per serving. For example, a single serving of a composition described herein can include cobalamin (vitamin B12) in an amount of about 0.0012 mg to about 0.0048 mg (e.g., about 0.002 mg, about 0.0024 mg, or about 0.0028 mg).

### Pantothenic Acid (Vitamin B5)

In some cases, the compositions described herein can include pantothenic acid (also known as vitamin B5). Pantothenic acid (also known as vitamin B5) can be present as D-pantothenic acid, dexpanthenol, and/or calcium pantothenate. In some cases, compositions described herein can include about 0.081% input of pantothenic acid. In some cases, compositions described herein can include about 0.00056% to about 0.56% (e.g., about 0.00056% to about 0.001%, about 0.001% to about 0.005%, about 0.005% to about 0.01%, about 0.01% to about 0.05%, about 0.05% to about 0.1%, about 0.1% to about 0.5%, about 0.5% to about 0.56%, or about 0.1% to about 0.56%) by weight of pantothenic acid (e.g., by weight of active vitamin B5 delivered). For example, a composition described herein can include about 0.05% to about 0.5% (e.g., about 0.055%, about 0.057%, about 0.075%, about 0.1%, about 0.25%, about 0.5%) by weight of pantothenic acid (e.g., by weight of active vitamin B5 delivered).

In some cases, compositions described herein can include pantothenic acid in an amount of about 0.05 mg to about 50 mg (e.g., about 0.05 mg to about 0.50 mg, about 0.50 mg to about 1 mg, about 1 mg to about 5 mg, about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg) per serving. For example, a single serving of a composition described herein can include pantothenic acid in an amount of about 2.5 mg to about 10 mg (e.g., about 4 mg, about 5 mg, or about 6 mg).

### Acidifiers

In some cases, the compositions described herein can contain one or more acidifiers. The acidifiers can modify the taste of the compositions. Acidifiers that can be used in the compositions include, without limitation, citric acid and malic acid. Additionally, or in the alternative, mineral or amino acid bound acids (e.g., potassium citrate and/or citrulline malate) can be used as acidifiers in the compositions provided herein.

In some cases, compositions described herein can include about 0.50% to about 50.0% (e.g., about 0.50% to about 1.0%, about 1.0% to about 5.0%, about 5.0% to about 10.0%, about 10.0% to about 15.0%, about 15.0% to about 20.0%, about 20.0% to about 25.0%, about 25.0% to about 30.0%, about 30.0% to about 35.0%, about 35.0% to about 40.0%, about 40.0% to about 45.0%, or about 45.0% to about 50.0%) by weight of one or more acidifiers.

In some cases, compositions described herein can include one or more acidifiers in an amount of about 10 mg to about 4000 mg (e.g., about 10 mg to about 50 mg, about 50 mg to about 100 mg, about 100 mg to about 150 mg, about 150 mg to about 200 mg, about 200 mg to about 250 mg, about 250 mg to about 300 mg, about 300 mg to about 350 mg, about 350 mg to about 400 mg, about 400 mg to about 450 mg, about 450 mg to about 500 mg, about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, about 950 mg to about 1000 mg, about 1000 mg to about 1500 mg, about 1500 mg to about 2000 mg, about 2000 mg to about 2500 mg, about 2500 mg to about 3000 mg, about 3000 mg to about 3500 mg, or about 3500 mg to about 4000 mg) per serving.

### Sweeteners

In some cases, the compositions described herein can contain one or more sweeteners. For example, the compositions can contain one or more of stevia, sucralose, acesulfame potassium, aspartame, monk fruit sugar, dextrose, sucrose, fructose, glucose, maltose, cane sugar, sugar alcohol (e.g., erythritol, maltitol, and/or xylitol), and/or rare sugars (e.g., allulose and/or tagatose).

In some cases, compositions described herein can include about 0.10% to about 40.0% (e.g., about 0.10% to about 0.50%, about 0.50% to about 1.0%, about 1.0% to about 2.0%, about 2.0% to about 3.0%, about 3.0% to about 4.0%, about 4.0% to about 5.0%, about 5.0% to about 6.0%, about 6.0% to about 7.0%, about 7.0% to about 8.0%, about 8.0% to about 9.0%, about 9.0% to about 10.0%, about 10.0% to about 15.0%, about 15.0% to about 20.0%, about 20.0% to about 25.0%, about 25.0% to about 30.0%, about 30.0% to about 35.0%, or about 35.0% to about 40.0%) by weight of one or more sweeteners.

In some cases, compositions described herein include one or more sweeteners in an amount of about 5 mg to about 30 g (e.g., about 5 mg to about 10 mg, about 10 mg to about 50 mg, about 50 mg to about 100 mg, about 100 mg to about 150 mg, about 150 mg to about 200 mg, about 200 mg to about 250 mg, about 250 mg to about 300 mg, about 300 mg to about 350 mg, about 350 mg to about 400 mg, about 400 mg to about 450 mg, about 450 mg to about 500 mg, about 500 mg to about 600 mg, about 600 mg to about 700 mg, about 700 mg to about 800 mg, about 800 mg to about 900 mg, about 900 mg to about 1 g, about 1 g to about 5 g, about 5 g to about 10 g, about 10 g to about 15 g, about 15 g to about 20 g, about 20 g to about 25 g, or about 25 g to about 30 g) per serving.

### Thickeners

In some cases, the compositions described herein can contain one or more thickeners. The thickeners can act as, for example, mouthfeel agents. Additionally, or in the alternative, thickeners can keep the active ingredients of the composition in suspension (e.g., in solution). Thickeners used in the compositions provided herein can include, without limitation, one or more of guar gum, xanthan gum, locust bean gum, and cellulose gum.

In some cases, compositions described herein can include about 0.10% to about 10.0% (e.g., about 0.10% to about 0.50%, about 0.50% to about 1.0%, about 1.0% to about 2.0%, about 2.0% to about 3.0%, about 3.0% to about 4.0%, about 4.0% to about 5.0%, about 5.0% to about 6.0%, about 6.0% to about 7.0%, about 7.0% to about 8.0%, about 8.0% to about 9.0%, or about 9.0% to about 10.0%) by weight of one or more thickeners.

In some cases, compositions described herein can include one or more thickeners in an amount of about 5 mg to about 500 mg (e.g., about 5 mg to about 10 mg, about 10 mg to about 50 mg, about 50 mg to about 100 mg, about 100 mg to about 150 mg, about 150 mg to about 200 mg, about 200 mg to about 250 mg, about 250 mg to about 300 mg, about 300 mg to about 350 mg, about 350 mg to about 400 mg, about 400 mg to about 450 mg, or about 450 mg to about 500 mg) per serving.

### Formulations

The dietary supplement compositions of the present disclosure can be formulated in any appropriate manner. For example, the compositions can be formulated as a solid (e.g., a powder). In some cases, the compositions can be formulated as one or more tablets, soft gelatin capsules, hard gelatin capsules, sachets, gels, confectionary suspensions, supplemented foods, or bars). In some cases, the compositions provided herein can be formulated as a liquid (e.g., a solution, shake, or syrup).

In some cases, the dietary supplement compositions provided herein can be formulated for oral administration and can include one or more suitable excipients, flavorings (e.g., one or more natural flavors), colorants (e.g., vegetable powder, vitamins, algae, spices, fruits, or other botanicals), and/or any other suitable ingredients. For example, compositions for oral administration, such as powders, tablets, or capsules, can be prepared with pharmaceutically acceptable excipients such as one or more binding agents, fillers, lubricants, disintegrants, or wetting agents. In some cases, dietary supplement compositions provided herein can contain a pharmaceutically acceptable carrier for administration to a subject (e.g., a mammal, such as a human), including, without limitation, sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Pharmaceutically acceptable carriers can also include physiologically acceptable aqueous vehicles (e.g., physiological saline) or other carriers appropriate for oral administration. In some cases, tablets can include a coating (e.g., a polymer or polysaccharide-based coating with or without plasticizers and/or pigments). Liquid preparations for oral administration can be in the form of, for example, solutions, syrups, or suspensions, or they can be presented as a dry product (e.g., a powder) for constitution with water or another suitable liquid (e.g., water) before use. In some cases, liquid preparations can contain pharmaceutically acceptable additives such as suspending agents, emulsifying agents, non-aqueous vehicles, preservatives, buffer salts, flavoring agents, coloring agents, and sweetening agents as appropriate. Preparations for oral administration can be formulated to give controlled release of one or more compounds. In some cases, tablets or capsules can be coated with a methacrylic acid copolymer and/or sodium alginate for release beyond the stomach (e.g., in the intestine, colon, or both). In some cases, tablets or capsules can be coated with sodium alginate for extension of release and better absorption (e.g., via liposomal technology). Other examples of slow release coatings that can be used include, without limitation, stearic acid, carbomer copolymer, shellac, hypromellose, carboxymethylcellulose sodium, carrageenan, cellaburate, ethylcellulose, glyceryl monooleate, pregelatinized modified starch, glyceryl monostearate, guar gum, hydroxypropyl betadex, hydroxypropyl cellulose, polyethylene oxide, polyvinyl acetate dispersion, pregelatinized starch, xanthan gum, alginic acid, locust bean gum, hydrogenated vegetable oil, monoglycerides, diglycerides, and combinations thereof.

In some cases, the composition can be formulated as a powder that can be mixed in a liquid beverage (including, but not limited to, water, milk, fruit juice, vegetable juice, tea, coffee) before consumption. In some cases, the composition can be formulated as a powder that can be directly consumed. In some cases, the composition can be formulated as a powder that can be mixed with one or more food items before consumption. In some cases, a serving of the dietary supplement composition can be about 6 g to about 12 g (e.g., about 6 g to about 8 g, about 7 g to about 9 g, about 8 g to about 10 g, about 9 g to about 11 g, about 10 g to about 12 g, or about 8 g to about 9 g) in powder form. In some cases, a serving of the dietary supplement composition can be about 8.8 g in powder form.

In some cases, the dietary supplement compositions provided herein can be in the form of capsules, tablets, or sachets of powder, by way of an example only, configured to have a unit dosage equal to the desired dosage per serving for a particular subject. For example, if a subject is to be administered a dose of 100 mg of the composition, each capsule or tablet or sachet of powder can include about 100 mg in weight of that composition. In some cases, a total dose per serving may be prepared and administered in the form of one or more dosage units (e.g., two tablets or capsules or sachets, three tablets or capsules or sachets, four tablets or capsules or sachets, five tablets or capsules or sachets, or six tablets or capsules or sachets). For instance, in some cases, an exemplary dietary supplement composition can be provided in three separate tablets or capsules or sachets. In some cases, the desired dosage (e.g., desired dosage per serving) of a composition can be included in a particular volume of the composition (e.g., in one or more teaspoons, tablespoons, scoops, or cups).

In some cases, a serving can be 8 ounces of a beverage dietary supplement composition or an amount of dry powder dietary supplement composition intended to be mixed with a liquid to form a single serving. For example, 8 ounces of water can be combined with about 6-12 g (e.g., about 6 g, about 7 g, about 7.5 g, about 8 g, about 8.5 g, about 9 g, about 9.5 g, about 10 g, about 11 g, or about 12 g) of a dry powder dietary supplement composition provided herein to form a beverage dietary supplement composition. In some cases, a container or package containing a dry powder dietary supplement composition provided herein can include a measuring scoop or spoon sized to measure out a single serving of a dry powder dietary supplement composition provided herein. For example, a package (e.g., a jar, pouch, or any other suitable container) can contain multiple servings of a dry powder dietary supplement composition provided herein, and can be provided to a subject in combination with a measuring spoon, a scoop, or any other device suitable for measuring out a single serving of a dry powder dietary supplement composition provided herein. In some cases, a single serving of a dry powder dietary supplement composition provided herein can be packaged in a single serving packet, pouch, or any other appropriate container from which the dry powder dietary supplement composition can be dispensed into a fluid (e.g., water or another beverage) before consumption. In some cases, a plurality of such single serving containers can be packaged together (e.g., in a box or a larger pouch). The compositions provided herein can be formulated for administration to any appropriate subject. An appropriate subject can be a human or another mammal. For example, a subject can be a human consumer in the fitness and wellness space. In some cases, the subject is a human who is undergoing physical training or who will be undergoing physical training.

### Uses as Dietary Supplements

The present disclosure also provides methods for using the compositions described herein as dietary supplements. For example, the compositions described herein can be used as pre-workout supplements (e.g., supplements used prior to physical exercise or physical training). In some cases, the compositions provided herein can be administered to a subject (e.g., can be consumed by a subject) about 1 minute to about 10 hours (e.g., about 1 minute to about 5 minutes, about 5 minutes to about 10 minutes, about 10 minutes to about 15 minutes, about 15 minutes to about 20 minutes, about 20 minutes to about 25 minutes, about 25 minutes to about 30 minutes, about 30 minutes to about 35 minutes, about 30 minutes to about 60 minutes, about 30 minutes to about 90 minutes, about 30 minutes to about 120 minutes, about 60 minutes to about 90 minutes, about 35 minutes to about 40 minutes, about 40 minutes to about 45 minutes, about 45 minutes to about 50 minutes, about 50 minutes to about 55 minutes, about 55 minutes to about 1 hour, about 1 hour to about 2 hours, about 2 hours to about 3 hours, about 3 hours to about 4 hours, about 4 hours to about 5 hours, about 5 hours to about 6 hours, about 6 hours to about 7 hours, about 7 hours to about 8 hours, about 8 hours to about 9 hours, or about 9 hours to about 10 hours) prior to a workout. In some cases, the compositions provided herein can be administered to a subject (e.g., can be consumed by a subject) during a workout.

The compositions can be administered to a subject (e.g., can be consumed by a subject) one time in a day. Alternatively, the compositions can be administered (e.g., consumed) more than one time (e.g., two times, three times, or four times) in one day. In some cases, the compositions can be administered (e.g., consumed) one or more times (e.g., two times, three times, four times, five times, six times, seven times, eight times, nine times, ten times, or more than ten times) in 2 days, 3 days, 4 days, 5 days, 6 days, 7 days, 1 week, 2 weeks, 3 weeks, 4 weeks, 1 month, 2 months, 3 months, 4 months, 5 months, 6 months, 7 months, 8 months, nine months, 10 months, 11 months, 1 year, or more.

The compositions described herein can be effective to increase energy and/or workout performance in a subject (e.g., a subject who consumed a composition provided herein prior to physical training), as compared to a control subject. A control subject can be one to whom a composition of the present disclosure has not been administered. In some cases, the control subject can be the same subject before consumption of a composition provided herein. The compositions described herein can increase energy and/or workout performance in a subject by about 1-fold or more (e.g., by about 2-fold, 3-fold, 5-fold, 10-fold, 15-fold, 20-fold, or more), as compared to a control subject. Increases in energy and/or workout performance can include, without limitation, one or more of the following in the subject: muscle activation, reduced oxygen cost, increased oxygen supply in muscles, increased oxygen supply in brain, increased cognitive function, increased attention, increased power, increased strength, increased endurance (e.g., cardiorespiratory endurance and/or muscle endurance), increased flexibility, increased muscle strength, improved body composition (e.g., improved percentages of fat, bone and/or muscle in the body of a subject, such as reduced body fat and/or increased lean muscle), reduced reaction time, and reduced fatigue. The increase in energy and/or workout performance can be acute, such as after a single consumption or administration of the composition (e.g., consumption or administration of a single dose or single serving of the composition). Additionally, or in the alternative, the increase in energy and/or workout performance can be longitudinal (e.g., may last for one or more days, weeks, or months), such as after repeated consumption or administration of the composition.

In some cases, changes in energy and/or work out performance can be measured by one or more of the following methods: measuring changes in strength (e.g., 1 repetition maximum (1RM), 3 repetition maximum (3RM), Cap at 5RM); measuring strength endurance, such as, repetitions to failure at a fixed load/intensity (e.g., 10RM, 15RM); measuring endurance (e.g., 5 km, 10 km, 20 km, 50 km, 100 km time trial; cyclists may go to 560 km; runners may go to 200 km); measuring aerobic capacity (e.g., maximum oxygen consumption (VO2max), peak oxygen consumption (VO2peak)); measuring anaerobic capacity (e.g., Maximal lactate steady state; 50 m, 100 m, 200 m, 400 m sprint times; repeated sprint performance; 500 m row); and/or measuring power (e.g., vertical jump height; bench press throw; force generation; torque).

The compositions and methods provided herein will be further described in the following examples, which do not limit the scope of the disclosure.

### EXAMPLES

### Example 1. An Exemplary Dietary Supplement Composition

A dietary supplement was prepared containing the ingredients shown in Table 1 below. The dietary supplement was prepared as an edible powder to be consumed after mixing with water or any other suitable beverage.

TABLE 1
Exemplary dietary supplement composition.

| Item Description | Amount (UM/Serving) |
|---|---|
| Mango leaf extract (60% mangiferin) | 30-3000 mg |
| Arugula extract (std. nitrate) | 30-4000 mg |
| Grape seed dry extract | 30-3000 mg |

### Example 2. An Exemplary Dietary Supplement Composition

A dietary supplement was prepared containing the ingredients listed in Table 2 below. The dietary supplement was prepared as an edible powder to be consumed after mixing with water or any other suitable beverage.

TABLE 2
Exemplary dietary supplement composition.

| Item Description | Amount (UM/Serving) | |
|---|---|---|
| Mango leaf extract (60% mangiferin) | 30-3000 | mg |
| Arugula extract (std. nitrate) | 30-4000 | mg |
| Grape seed dry extract | 30-3000 | mg |
| Caffeine (coffee bean 98%) | 20-800 | mg |

### Example 3. An Exemplary Dietary Supplement Composition

A dietary supplement was prepared containing the ingredients listed in Table 3 below. The dietary supplement was prepared as an edible powder to be consumed after mixing with water or any other suitable beverage.

TABLE 3
Exemplary dietary supplement composition.

| Item Description | Amount (UM/Serving) | |
|---|---|---|
| Mango leaf extract (60% mangiferin) | 30-3000 | mg |
| Arugula extract (std. nitrate) | 30-4000 | mg |
| Grape seed dry extract | 30-3000 | mg |
| B6 HCl | 0.05-5 | mg |
| Thiamin HCl | 0.01-1 | mg |
| Riboflavin | 0.01-1 | mg |
| Niacinamide | 1-100 | mg |
| Vitamin B12 | 0.0002-0.02 | mg |
| Vitamin B5 | 0.05-50 | mg |

### Example 4. An Exemplary Dietary Supplement Composition

A dietary supplement was prepared containing the ingredients listed in Table 4 below. The dietary supplement was prepared as an edible powder to be consumed after mixing with water or any other suitable beverage.

TABLE 4
Exemplary dietary supplement composition.

| Item Description | Amount (UM/Serving) | |
|---|---|---|
| Mango leaf extract (60% mangiferin) | 30-3000 | mg |
| Arugula extract (std. nitrate) | 30-4000 | mg |
| Grape seed dry extract | 30-3000 | mg |
| Caffeine (coffee bean 98%) | 20-800 | mg |
| B6 HCl | 0.05-5 | mg |
| Thiamin HCl | 0.01-1 | mg |
| Riboflavin | 0.01-1 | mg |
| Niacinamide | 1-100 | mg |
| Vitamin B12 | 0.0002-0.02 | mg |
| Vitamin B5 | 0.05-50 | mg |

### Other Embodiments

It is to be understood that the foregoing description is intended to illustrate and not limit the scope of the disclosure, which is defined by the scope of the appended claims. Other aspects, advantages, and modifications are within the scope of the following claims.

What is claimed is:

1. A composition comprising:
arugula extract;
grape seed extract, grape skin extract, or a combination thereof; and
mango leaf extract.

2. The composition of claim 1, wherein the composition comprises about 20% to about 50% by weight of said arugula extract.

3. The composition of claim 1, wherein the composition comprises said arugula extract in an amount sufficient to produce about 90 mg to about 1240 mg nitrate.

4. The composition of claim 1, wherein the composition comprises said arugula extract in an amount of about 2 g to about 15 g.

5. The composition of claim 1, wherein the composition comprises about 2% to about 5% by weight of said grape seed extract, grape skin extract, or combination thereof.

6. The composition of claim 1, wherein the composition comprises said grape seed extract, grape skin extract, or combination thereof in an amount of about 100 mg to about 500 mg.

7. The composition of claim 1, wherein the composition comprises about 2% to about 5% by weight of said mango leaf extract.

8. The composition of claim 1, wherein the composition comprises said mango leaf extract in an amount of about 140 mg to about 19,350 mg.

9. The composition of claim 1, further comprising caffeine.

10. The composition of claim 9, wherein the composition comprises said caffeine in an amount of about 18 mg to about 540 mg.

11. The composition of claim 1, further comprising at least one B vitamin.

12. The composition of claim 11, wherein the composition comprises one or more of:
about 0.01% to about 0.05% by weight of thiamin,
about 0.01% to about 0.05% by weight of riboflavin,
about 0.05% to about 0.25% by weight of niacin,
about 0.005% to about 0.05% by weight of B6,
about 0.001% to about 0.01% by weight of B12, and
about 0.00056% to about 0.56% by weight of pantothenic acid.

13. The composition of claim 11, wherein the composition comprises one or more of:
thiamin in an amount of about 0.12 mg to about 12 mg,
riboflavin in an amount of about 0.13 mg to about 13 mg,
niacin in an amount of about 1.6 mg NE to about 80 mg NE,
B6 in an amount of about 0.17 mg to about 170 mg,
B12 in an amount of about 0.00024 mg to about 4.8 mg, and
pantothenic acid in an amount of about 0.05 mg to about 50 mg.

14. The composition of claim 1, wherein the composition comprises:
(a) arugula extract;
(b) grape seed extract, grape skin extract, or combination thereof;
(c) mango leaf extract;
(d) caffeine; and
(e) one or more B vitamins.

15. The composition of claim 1, further comprising at least one acidifier, at least one sweetener, a thickener, one or more natural flavors, one or more colorants, or any combination thereof.

16. The composition of claim 1, wherein the composition is formulated as a solid.

17. The composition of claim 1, wherein the composition is formulated as a liquid.

18. A method for increasing energy and workout performance in a subject, the method comprising administering to the subject the composition of claim 1.

19. The method of claim 18, wherein the method comprises activating muscles in the subject.

20. The method of claim 18, wherein the composition is effective to reduce oxygen cost and increase oxygen supply in muscles and brain of the subject.

21. The method of claim 18, wherein the composition is administered to the subject prior to physical training.

22. The method of claim 18, wherein the composition is administered to the subject during physical training.

23. A powdered dietary supplement composition contained in a bulk package, the bulk package containing a measuring scoop and the powdered dietary supplement composition, the measuring scoop being sized to scoop an amount of the powdered dietary supplement composition suitable for blending with a liquid to form a beverage, wherein the amount of the powdered dietary supplement composition is 15 g to 40 g and comprises:
(a) arugula extract in an amount of about 20 wt % to about 50 wt %;
(b) grape seed extract, grape skin extract, or a combination thereof, in an amount of about 2 wt % to about 5 wt %; and
(c) mango leaf extract in an amount of about 2 wt % to about 5 wt %.

24. The powdered dietary supplement composition of claim 23, further comprising caffeine in an amount of about 1 wt % to about 5 wt %.

25. The powdered dietary supplement composition of claim 23, further comprising at least one B vitamin.
